(19) United States
(12) Patent Application Publication
(10) Pub. No.: US 2005/0257295 A1
(43) Pub. Date: Nov. 17, 2005

(54) Production of reticuline

(76) Inventors: Anthony John Fist, Norwood (AU); Christopher James Byme, Westbury (AU); Wayne Lyle Gerlach, Killara (AU); Christopher Charles Sayer, Summerhill (AU); Timothy Samuel Bailey, Westbury (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/186,508

(22) Filed: Jul. 21, 2005

Related U.S. Application Data
(63) Continuation of application No. 10/993,527, filed on Nov. 18, 2004.
Application No. 10/993,527 is a continuation of application No. 09/600,500, filed on Nov. 15, 2000, now abandoned.

(30) Foreign Application Priority Data
Jan. 14, 1998 (AU) PP 1321

(51) Int. Cl. A01H 5/00

(52) U.S. Cl. 800/295

(57) ABSTRACT

The present invention is concerned with methods for improved production of reticuline. More particularly, the present invention relates to the use of a mutagenized Papaver somniferum poppy plant to produce (S)-reticuline in higher yield. The invention also relates to methods for extracting and purifying reticuline.

### TECHNICAL FIELD

The present invention relates to the improved production of reticuline. More particularly, the present invention relates to the use of a mutagenized Papaver somniferum poppy plant to produce (S)-reticuline in higher yield. The invention also relates to methods of extracting and purifying reticuline.

### BACKGROUND OF THE INVENTION

(S)-Reticuline is an intermediate in the biosynthetic pathway leading to phenanthrene alkaloids such as codeine and morphine, phthalidisoquinoline alkaloids such as noscapine and benzylisoquinoline alkaloids such as papaverine in the Papaver somniferum poppy (Scheme 1). (S)-Reticuline is present in other plants, such as Eschscholzia californica, Corydalis cava, Fumaria officinalis, Berberis vulgaris and Sanguinaria canadensis, and has been identified as a precursor of protopine, benzo[c]phenanthridine alkaloids such as sanguinarine, protoberberine alkaloids such as corydaline and berberine itself.

These compounds are pharmaceutically useful, for example, the analgesic properties and commercial value of codeine and morphine require little introduction. Noscapine is a useful antitussive compound. Papaverine is a smooth muscle relaxant and a cerebral vasodilator. Berberine has been used as an antibacterial, antimalarial and antipyretic compound.

As well as being an important precursor for numerous pharmaceutical products, (S)-reticuline has recently been shown to accelerate hair growth in cultured hair cells (Biol. Pharm. Bull., 20(5)586-588 (1997)).

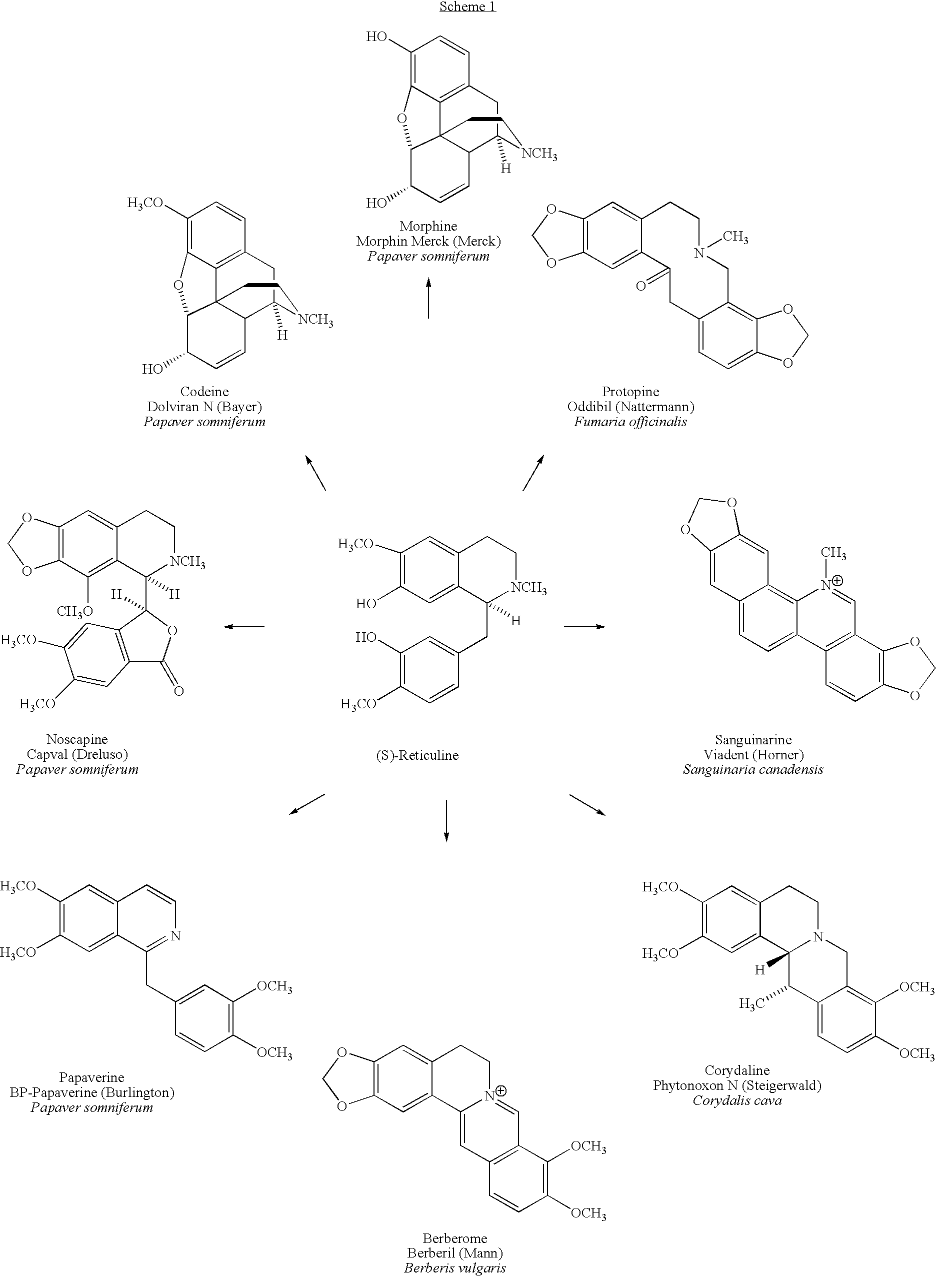

(±)-Reticuline has been synthesised, by a lengthy and difficult synthesis (Tomita, M. and Kikkawa, I., Pharm Bull Japan, 4, 230 (1956), Chem Abs, 51, 8116 (1957) and Gopinath K. W., Govindachari, T. R., and Viswanathan N. Ber, 92. 1657 (1959)).

The synthesis of the (S) form has also been reported by Konda et al. Chem Pharm Bull, 23, 1063 (1975). Whilst effective, the difficulty of the totally synthetic route is that only small quantities of the compound are available after a long and costly synthesis. Thus, total synthesis is undesirable as a means of making substantial quantities of (S)-reticuline.

A second reason for the limited availability and high cost of (S)-reticuline is that it is present in source plants at very low concentrations. For instance it is found in commercial poppy straw at 0.04%, and it is present in the opium of Papaver somniferum in trace amounts (Brochman-Hanssen, E. and Furaya, T., Planta Med. 12, 328 (1964)). Due to the low concentrations of (S)-reticuline in the various plant sources, there is at present no commercial source of (S)-reticuline.

(S)-Reticuline has been isolated from opium by conventional but lengthy extraction procedures. The initial step involves the mixing of powdered opium with a cationic exchange resin in hot water. The alkaloids adsorb to the ion exchange resin and the non polar fractions which are not of interest are removed by washing. The alkaloid fractions are removed by elution with methanol and can be extracted into organic solvents, such as chloroform, by using controlled acid/base extractions: for example, see the work by Brochmann-Hanssen and Furuya, 1964, Planta Med. 12, 328 and references cited therein.

Such an extraction process is expensive and involve considerable losses of opium derived material. The yield of (S)-reticuline from opium is low, Brochmann-Hanssen and Furuya reporting that it represents about 0.15% of the total opium mass. These factors all combine to render (S)-reticuline extraction from opium commercially unattractive.

Alkaloids are extracted from the poppy capsules of Papaver somniferum by two commercial methods. In one method, the immature capsule is cut and the latex collected from the wound and air dried to produce opium. In a second method, the mature poppy capsules and the poppy capsule stems are collected, and threshed to remove the seeds and form a straw. When necessary, the straw is dried to a water content below 16%. Solvent or water extraction is employed to remove the alkaloids from the straw.

Where solvent, water or super critical fluid, such as CO₂, extraction is employed to remove the phenanthrene alkaloids from the straw, such method, as practiced, involves the production of “Concentrate of Poppy Straw”. Concentrate of poppy straw has been defined as “The material arising when poppy straw has entered into a process for the concentration of its alkaloids, when such material is made available in trade (Multilingual Dictionary of Narcotic Drugs and Psychotropic Substances Under International Control, United Nations, New York, 1983). Concentrate of poppy straw is also defined as “the crude extract of poppy straw in either liquid, solid or powder form which contains the phenanthrene alkaloids of the opium poppy” 45 U.S. Federal Register 77466, Nov. 24, 1980. For the purposes of the present specification, the term “extracted alkaloid mixture” will be used to define the crude extract extracted from poppy straw, which may contain benzylisoquinoline alkaloids, phthalidisoquinoline alkaloids and/or phenanthrene alkaloids. The “extracted alkaloid mixture” is taken to mean the crude extract of poppy straw in either liquid solid or powder form. When in liquid form, the liquid is preferably concentrated before entering commerce. The generally preferred extracted alkaloid mixture is the powder form which results form simply removing the solvent or water following extraction of the poppy straw.

As the synthesis of (S)-reticuline is economically impractical, and extraction from natural sources is low yielding and requires extensive purification, it would be desirable to increase production by increasing the amount of (S)-reticuline produced by a plant.

It is also desirable to increase the ratio of (S)-reticuline to phenanthrene-type alkaloids in the plant and the plant products. Phenanthrene alkaloids are those incorporating the phenanthrene ring system into their structure. Morphine, codeine, thebaine and oripavine are examples of such a phenanthrene type alkaloid. Reticuline however does not include this structural element but instead is based on benzyl-isoquinoline as its major structural element.

Surprisingly, the present inventors have found a method of increasing (S)-reticuline production and the (S)-reticuline to phenanthrene alkaloid ratio by modifying Papaver somniferum.

It is an object of the present invention to provide a commercially viable alternative to the methods in the prior art.

It will be understood by a skilled addressee that the present invention, whilst exemplified in relation to Papaver somniferum, would be equally applicable to other plants in which (S)-reticuline is present, such as Eschscholzia californica. Corydalis cava, Fumaria officinalis, Berberis vulgaris and Sanguinaria canadensis.

In the context of the present invention, the term “opium” is taken to include material which is obtained from a modified Papaver somniferum in a similar fashion to that used to obtain opium (as conventionally defined) from a non-modified plant.

### SUMMARY OF THE INVENTION

In a first aspect the invention provides a stably reproducing Papaver somniferum having an (S)-reticuline content higher than that of a native Papaver somniferum.

In a second aspect the invention provides a stably reproducing Papaver somniferum, which upon the harvesting of the poppy capsules will yield a poppy straw having an (S)-reticuline content higher than the poppy straw obtained from a native Papaver somniferum.

In a third aspect the invention provides a stably reproducing Papaver somniferum, which upon the collection and drying of the latex from the immature poppy capsules will yield an opium having an (S)-reticuline content higher than the latex obtained from a native Papaver somniferum.

In a preferred embodiment the production or activity of (S)-reticuline oxidase in the stably reproducing Papaver somniferum is inhibited, with the result that upon harvesting the poppy capsules will yield a poppy straw, or upon the collection and drying of the latex from the immature poppy capsules will yield an opium, having a (S)-reticuline content higher than the poppy straw of a native Papaver somniferum.

In another preferred embodiment the production or activity of dehydroreticuline reductase in the stably reproducing Papaver somniferum is inhibited, with the result that upon harvesting the poppy capsules will yield a poppy straw or upon the collection and drying of the latex from the immature poppy capsules will yield an opium, having a (S)-reticuline content higher than the poppy straw of a native Papaver somniferum. In yet another preferred embodiment the production or activity of berberine bridge enzyme (BBE) in the stably reproducing Papaver somniferum is inhibited, with the result that upon harvesting the poppy capsules will yield a poppy straw, or upon the collection and drying of the latex from the immature poppy capsules will yield an opium, having a (S)-reticuline content higher than the poppy straw of a native Papaver somniferum.

In a further preferred embodiment the production or activity of two or more enzymes in a stably reproducing Papaver somniferum, selected from the group comprising: (S)-reticuline oxidase dehydroreticuline reductase or berberine bridge enzyme (BBE), are inhibited with the result that upon harvesting the poppy capsules will yield a poppy straw, or upon the collection and drying of the latex from the immature poppy capsules will yield an opium, having a (S)-reticuline content higher than the poppy straw of a native Papaver somniferum.

Preferably, such stably reproducing Papaver somniferum yield a poppy straw having an (S)-reticuline content greater than 1.0%, and more preferably greater than 2.5%.

Preferably, such stably reproducing Papaver somniferum yield opium having an (S)-reticuline content greater than 10%, and more preferably greater than 20%.

Preferably, such stably reproducing Papaver somniferum yields an extracted alkaloid mixture having an (S)-reticuline content greater than 30%, and more preferably greater than 60%.

Also preferred is a stably reproducing Papaver somniferum which upon the harvesting of the poppy capsules will yield a poppy straw, an opium or an extracted alkaloid mixture having an (S)-reticuline to phenanthrene alkaloid ratio of about 100% or greater. More preferred is a ratio of 200% or greater, even more preferred is a ratio of 1250% or greater and highly preferred is a ratio of about 2500%. In yet another preferred embodiment a stably reproducing Papaver somniferum, upon the harvesting of the poppy capsules will yield a poppy straw, an opium or an extracted alkaloid mixture having substantially no phenanthrene alkaloid content.

According to a fourth aspect the invention provides a seed yielding a stably reproducing Papaver somniferum according to any one of the preceding aspects.

According to a fifth aspect the invention provides poppy straw of a stably reproducing Papaver somniferum, the threshed straw having an (S)-reticuline content higher than that of the poppy straw of a native Papaver somniferum. Preferably, the poppy straw has an (S)-reticuline content greater than 1.0%, more preferably greater than 2.0%, even more preferably the (S)-reticuline content is about 3-4%.

Also preferred is poppy straw having (S)-reticuline to phenanthrene alkaloid ratio of 100% or greater by weight. More preferred is a ratio of 200% or greater by weight, even more preferred is a ratio of 1250% or greater by weight and highly preferred is a ratio of about 2500%. In a further preferred embodiment the poppy straw has substantially no phenanthrene alkaloid content.

According to a sixth aspect the invention provides opium of a stably reproducing Papaver somniferum, the opium having an (S)-reticuline content higher than that of the opium of a native Papaver somniferum. Preferably, the opium has an (S)-reticuline content greater than 10% and more preferably greater than 20%.

Also preferred is opium having (S)-reticuline to phenanthrene alkaloid ratio of 100% or greater by weight. More preferred is a ratio of 200% or greater by weight, even more preferred is a ratio of 1250% or greater by weight and highly preferred is a ratio of about 2500%. In a further preferred embodiment the opium has substantially no phenanthrene alkaloid content.

According to a seventh aspect the invention provides an extracted alkaloid mixture of a stably reproducing Papaver somniferum, the extracted alkaloid mixture having an (S)-reticuline content higher than that of the extracted alkaloid mixture of a native Papaver somniferum. Preferably, the extracted alkaloid mixture has an (S)-reticuline content greater than 30% and more preferably greater than 60%.

Also preferred is an extracted alkaloid mixture having (S)-reticuline to phenanthrene alkaloid ratio of 100% or greater by weight. More preferred is a ratio of 200% or greater by weight, even more preferred is a ratio of 1250% or greater by weight and highly preferred is a ratio of about 2500%. In a further preferred embodiment the extracted alkaloid mixture has substantially no phenanthrene alkaloid content.

According to an eighth aspect the invention provides a stand of a stably reproducing Papaver somniferum according to any one of the previous aspects.

According to a ninth aspect the invention provides (S)-reticuline when obtained from a stably reproducing Papaver somniferum, the poppy straw, the opium or an extracted alkaloid mixture, according to any one of the previous aspects.

According to a tenth aspect the invention provides a method for the production of (S)-reticuline which comprises the steps of:
a) harvesting poppy capsules of a stably reproducing Papaver somniferum to produce a straw where the straw has a higher (S)-reticuline content than the straw of a native Papaver somniferum, and
b) chemically extracting the (S)-reticuline from the straw.

According to an eleventh aspect the invention provides a method for the production of (S)-reticuline which comprises the steps of:
a) collecting and drying the latex of the immature poppy capsules of a stably reproducing Papaver somniferum to produce opium where the opium has a (S)-reticuline content higher than that of the opium of a native Papaver somniferum, and
b) chemically extracting the (S)-reticuline from the opium.

Preferably, in such methods, stably reproducing Papaver somniferum yield a poppy straw having an (S)-reticuline content greater than 1.0%, more preferably greater than 2.0%, even more preferably the (S)-reticuline content is about 3-4%.

Preferably, in such methods stably reproducing Papaver somniferum yield an opium having an (S)-reticuline content greater than 10%, and more preferably greater than 20%.

The invention also consists in (S)-reticuline when obtained by any of the forgoing processes.

According to a twelfth aspect the invention provides a method to improve the (S)-reticuline yield of a stably reproducing Papaver somniferum, the method comprising the steps of:
a) exposing at least one poppy-seed of Papaver somniferum to a mutagenizing agent,
b) growing the at least one poppy seed to produce a plant bearing a leaf or an immature poppy capsule, optionally through multiple self fertilized generations,
c) sampling the leaf or poppy capsule for the presence of (S)-reticuline, morphine and codeine, and
d) repeating steps a) to c) until a poppy plant of Papaver somniferum is obtained having a (S)-reticuline content higher than that of a native Papaver somniferum.

Preferably steps a) to c) are repeated until the (S)-reticuline content shows no further increase on mutagenesis.

According to a thirteenth aspect there is provided a method for the production of (S)-reticuline which comprises the steps of:
a) harvesting poppy capsules of a stably reproducing Papaver somniferum to produce a straw wherein the straw has an (S)-reticuline to phenanthrene alkaloid ratio of 100% or greater by weight, and
b) chemically extracting the (S)-reticuline from the straw.

According to a fourteenth aspect there is provided a method for the production of (S)-reticuline which comprises the steps of:
a) collecting and drying the latex of the immature poppy capsules of a stably reproducing Papaver somniferum to produce opium wherein the opium has an (S)-reticuline to phenanthrene alkaloid ratio of 100% or greater by weight, and
b) chemically extracting the (S)-reticuline from the opium.

According to a fifteenth aspect there is provided a method to improve the (S)-reticuline yield of a stably reproducing Papaver somniferum, the method comprising the steps of:
a) exposing at least one poppy seed of Papaver somniferum to a mutagenizing agent,
b) growing the at least one poppy seed to produce a plant bearing a leaf or an immature poppy capsule, optionally through multiple self fertilized generations,
c) sampling the leaf or poppy capsule for the presence of (S)-reticuline, morphine and codeine, and
d) repeating steps a) to c) until a poppy plant of Papaver somniferum is obtained having an (S)-reticuline to phenanthrene alkaloid ratio of 100% or greater by weight.

Preferably in the aforementioned products and methods, the (S)-reticuline to phenanthrene alkaloid ratio is 200% or greater by weight, even more preferably the ratio is 1250% or greater and highly preferred is a ratio of about 2500%.

It is also highly preferred that there are substantially no phenanthrene alkaloids present.

The invention also consists in (S)-reticuline when obtained from any of the forgoing plants or plant products.

According to a sixteenth aspect there is provided a method for purifying reticuline from an aqueous extract of poppy straw comprising the following steps:
(i) mix said extrast with toluene at near neutral pH and separate the aqueous and the non-aqueous phases,
(ii) mix aqueous phase from step (ii) with toluene at pH of about 8.5 to about 9.5 and separate the aqueous and the non-aqueous phases,
(iii) extract reticuline from the non-aqueous phase by caustic extraction.

Preferably the method further comprises the steps of (iv) mixing caustic extract of step (iii) with toluene at alkaline pH and separating the aqueous and the non-aqueous phases, (v) mixing the non-aqueous phase from step (iv) with water at acidic pH, and separating the aqueous and the non-aqueous phases, (vi) adding alkali to aqueous phase at ambient temperature, ageing for a time sufficient to induce formation of a precipitate and collecting precipitate containing reticuline.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

Those skilled in the art will appreciate also that there are other methods of affecting the targeted enzymes to increase the accumulation of (S)-reticuline, such as transfection and targeting of genes and/or m-RNA encoding the production of (S)-reticuline oxidase, dihydroreticuline reductase and berberine bridge enzyme (BBE).

### BRIEF DESCRIPTION OF FIGURES

FIG. 1 shows a HPLC trace of an extract of modified Papaver somniferum (bottom line) and an extract spiked with a standard for alkaloid analysis.

### DETAILED DESCRIPTION OF THE INVENTION

Utilizing the mutagenized plants of Papaver somniferum as described herein, persons skilled in the art easily know how to grow and reproduce such plants, collect the latex or the dried straw and purify the (S)-reticuline. As one embodiment of the present invention, seeds to the mutagenized plants of Papaver somniferum, as described herein, have been deposited under the Budapest Treaty with The American Type Culture Collection. 12301 Parklawn Drive, Rockville, Md. 20852, United States of America on ______, under Accession No ______, and will be made available upon the maturation of this application into a patent. The availability of these seeds is not to be construed as a license to practice this invention in contravention of rights granted under the authority of any government in accordance with its patent or breeder's rights laws.

Methods of seed mutagenesis as well as mutagens suitable for use in these methods, such as, ethyl methanesulfonate (EMS), are described in the Manual on Mutation Breeding, 2nd ed., I.A.E.A., Vienna 1977 or in Plant Breeding, Principles and Prospects, Chapman and Hall, London 1993. For X-ray mutagenized seeds, hydrated seeds might be treated with 20,000 rads, (30 cm from the source for 45 minutes using a filter). X-ray mutagenesis is described and compared to EMS mutagenesis by Filippetti, A. et al., “Improvement of Seed Yield in Vici Baba L. By Using Experimental Mutagenesis II Comparison of Gamma-Radiation and Ethyl-MethaneSulphonate (EMS) in Production of Morphological Mutants”, Euphytica 35 (1986) 49-59. DEB, diepoxybutane, mutagenized seeds might be obtained by soaking the seeds in water overnight, then soaking in 22 mM DEB for 4 hours, followed by extensive washing. Further mutagens include ethyl-2-chloroethyl sulphide, 2-chloroethyl-dimethylamine, ethylene oxide, ethyleneimine, dimethyl sulphonate, diethyl sulphonate, propane sulphone, beta-propiolactone, diazomethane, N-methyl-N-nitrosourethane, acridine orange and sodium azide. The preferred mutagen employed herein is EMS.

Mutagenesis utilizing EMS is well described in the literature. The Manual on Mutation Breeding, supra, reports a preferred EMS mutagenesis process for barley seeds as practiced by K. Mikaelson. In this preferred process, the seeds are prepared, pre-soaked, treated with the mutagen and post-washed.

In the preparation, uniform size seeds are selected and placed in mesh polyethylene bags, about 200 seeds. Subsequently, the seeds are kept in a dessicator over a 60% glycerol solution, which gives the seeds a moisture content of about 13%. In pre-soak, the seed bags are transferred to beakers with distilled or deionized water and soaked for 16-20 hours at a temperature of 20-22° C. The pre-soak period is important to the uptake or diffusion of mutagen. The pre-soak should be sufficient to promote diffusion of the mutagen into the seed and at the same time stimulate the embryo meristem tissue to start DNA synthesis. It is at this point that high mutation frequency can be achieved with minimal chromosome damage. To treat with the mutagen, the seed bags are transferred to beakers containing a solution of EMS in distilled or deionized water. For barley and wheat the maximal mutation frequencies are obtained under treatment conditions where the EMS concentration is 0.05-0.1 M, the bath temperature is 30-35° C., and the exposure time of the seeds to the bath is 0.5-2 hours. Relatively weak treatments are preferred in mass screening to achieve maximal mutation with minimal physiological damage. Such treatments give better germinability and survival, less plant growth reduction and less sterility compared with stronger treatments. A thorough post-wash in water after the EMS treatment is essential. This post-wash can be carried out in running tap water, preferably at not less than 15° C., for a period of not less than 4 hours. The EMS should be removed by the post-wash in order to prevent uncontrollable after-effects by the mutagen. After post-washing, the seeds should be planted as soon as possible. If the seeds cannot be planted soon after the mutagenesis process, they should be immediately dried back to a moisture content of about 13%.

This can be accomplished by simply air drying the seeds at room temperature and a reasonably low relative humidity.

Persons skilled in the art will recognize that this preferred mutagenesis method for barley and wheat seeds can be easily modified for poppy seeds. In the case of poppy seeds, it has been found useful and convenient by the inventors hereof to dispense with dessication, to extend the time of pre-soak to up to 48 hours and to lower the bath temperature of mutagen treatment to 20° C. Other modifications will be apparent to skilled practitioners.

After the seeds have been exposed to the mutagen, the seeds are grown to maturity in controlled conditions and self-pollinated. The seeds from the mature plant are taken and at least one seed is planted to grow an M2 generation. The M2 generation is screened for alkaloid production. Of course, it is possible to screen the M1 generation, but there are several advantages to screening the M2 generation. Firstly, screening the M2 generation insures that the trait resulting from mutagenesis can be inherited. Secondly, by growing the M2 generation, the basic hardiness of the plant is proven before screening. Thirdly, traits resulting from mutagenesis are generally inherited as recessive genes, and these will be homozygous in the M2 generation, i.e., and codeine. Despite the fact that definite answers are not now available, there are good reasons to believe that the correct answer is known.

A postulated biosynthitic pathway in Papaver somniferum via (S)-reticuline to morphine is shown in Scheme 2 below.

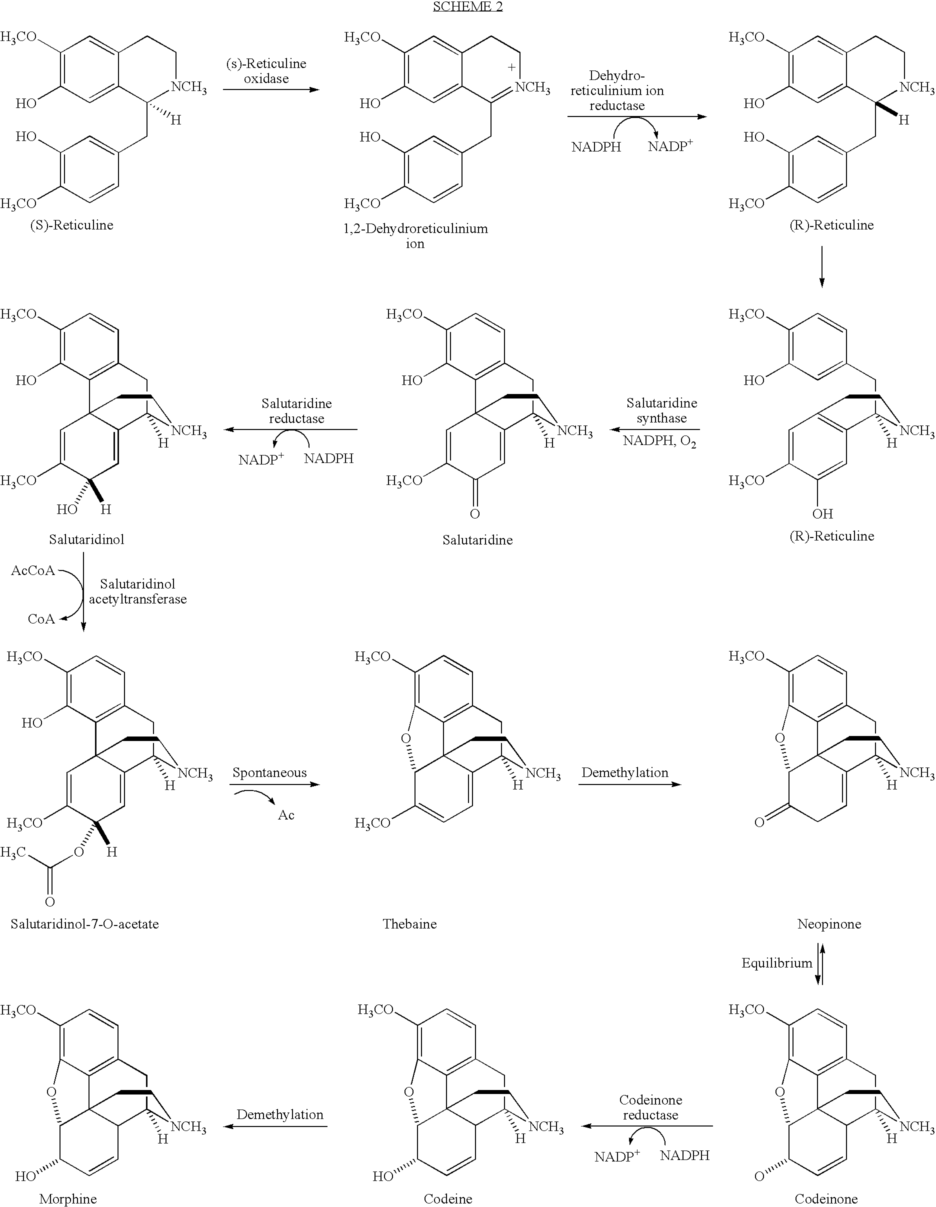

By the methods herein, a variety of Papaver somniferum was obtained having a high (S)-reticuline content and substantially no thebaine, codeine or morphine. Thus, it is believed, for the Papaver somniferum variety described herein, that the production or activity of (S)-reticuline oxidase has been substantially inhibited, resulting in a buildup of (S)-reticuline and less material following the biosynthetic pathway to its endpoint, i.e. morphine. It is also possible that the production or activity of dehydroreticuline reductase has been inhibited. By feedback inhibition through 1,2-dehydroreticuline, this would lead to an accumulation of (S)-reticuline.

It is also possible that stably reproducing Papaver somniferum in accordance with the present invention may also be obtained by recombinant DNA techniques. In particular, after isolation and sequencing of the gene coding for (S)-reticuline oxidase, the gene or the mRNA transcript may be modified, deleted or blocked to inhibit or prevent the production of (S)-reticuline oxidase. Techniques for modifying or deleting specific regions of DNA sequences or blocking mRNA are well known to those skilled in the art.

It would also be possible to accumulate (S)-reticuline in other species by blocking particular enzymes. For example, in Berberis species, the berberine bridge enzyme could be blocked either using mutagenesis (as demonstrated here) or through recombinant DNA techniques.

Recovering (S)-reticuline from either the dried straw or from the opium of Papaver somniferum is a process well established in the art. A schematic diagram (Scheme 3) is shown outlining the process of (S)-reticuline extraction from the alkaloid containing extract of opium. This procedure was outlined by Brochmann-Hanssen and Furuya (Planta Med. 12, 328-333). Methods of obtaining of a highly acidic (pH 1.5) opium extract are well known in the art. Those skilled in the art will appreciate that presently there are a number of suitable starting materials for such extractions depending on the industrial process being used, and that Scheme 3 provides one example only.

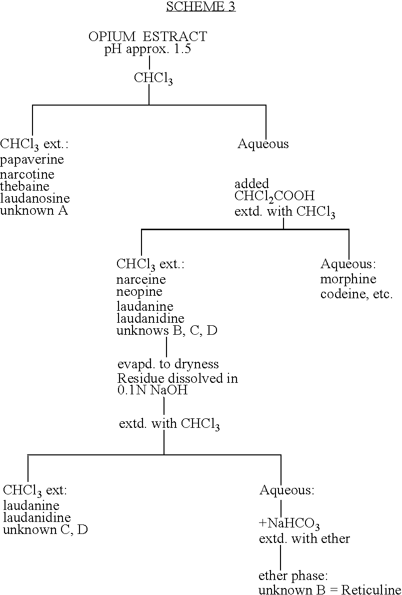

### EXAMPLES

### Example 1

### Mutation

Seeds of Papaver somniferum were obtained of about uniform size, dried to about 8% LOD (loss on drying) and placed in a mesh polyethylene bag at a weight of about 5 grams or about 12.500 per bag. The seeds were pre-soaked in beakers of distilled water containing a phosphate buffer at room temperature for about 36 hours. The seeds were given a further presoaking in cold 0.3% v/v (−0.028 M) ethyl methanesulphonate (EMS). Immediately after pre-soak, the seed bags were immersed in a mutagen bath containing 0.3% v/v (−0.028 M) ethyl methanesulphonate (EMS) at 20° C. for 6 hours. Immediately following the mutagen bath, the seed bags were post-washed in running tap water. Following post-wash, the seeds were kept moist and planted within one hour.

### Example 2

### Propagation

The seeds were planted in outdoor plots and grown to maturity. The planting technique employed was in all respects normal for poppy trial work, and similar to commercial poppy growing. The seeds were sown using a “cone seeder” or trial plot drill. Seed depth was about 1 cm. Fertiliser containing N, P and K was used. The plots were irrigated immediately after sowing. The poppy flowers were self-pollinated and the majority of the flowers were covered with paper bags of bleached white “kraft” paper to prevent cross pollination. Seeds were harvested from those M1 generation plants which grew vigorously and appeared healthy. A second, M2, generation was grown from the harvested seeds. These seeds were planted in trays containing 200 plants. When the M2 plants were between the 10 leaf stage and the “running-up” stage, about 15 cm high, they were screened for alkaloid content using a rapid HPLC technique.

### Example 3

### Screening

The screening process was basically a three step process. In the first step, a leaf was cut from an M2 plant and about 0.5 μL of latex was collected at the wound. The latex was diluted in a microcentrifuge tube with 250 mL of buffer. The buffer contained 0.2 M ammonium phosphate, 20% ethanol, and had a pH of 4.5. The microcentrifuge tube was briefly held to a vortex shaker to ensure mixing. In the second step, the buffered solution was centrifuged to substantially eliminate suspended solids and about 200 μL was decanted into a 40 mm×8 mm autoanalyser tube. Additional buffer, 250 μL, was added to each autoanalyser tube so that the sampling needle of the autoanalyser could reach the solution. In the third step, the autoanalyser tubes were loaded into a 96 place carousel inserted into the auto injector module of a Waters HPLC system. The HPLC mobile phase was aqueous methanol (approximately 30%) containing ammonium acetate buffer (0.08-0.12 M), pH 4-5. The flow rate of the mobile phase was 0.8-1.5 mL/minute. A Whatman Partisphere SCX column (4.6×125 mm) was used at a temperature of 40° C. A Waters 440 UV detector was used to detect the peaks at 254 nm. The data was interpreted and collated on a Waters Millennium Data Station. The system was used to analyse for alkaloids.

Two plants E40 and E41, were screened and the latex was found to be morphine and thebaine free and contained a peak later identified as (S)-reticuline. The two plants were combined and about 0.15 g of straw was harvested and analysed. The (S)-reticuline content was 3.3%, with 0.007% thebaine. The reticuline was identified by circular dichroism as (S)-reticuline.

A descendant generation was grown in the field. The plants grew well, but two distinct types of plant were observed at the green capsule stage, those having white latex (E40/41 W) and those having red latex (E40/41 R). From the variety with white latex was harvested 50.7 g of straw containing 3.88% (S)-reticuline and 0.78% codeine (or codeine-like alkaloids). The variety with red latex was observed to have 2.51% (S)-reticuline and zero codeine.

### Example 4

### Extraction

An acidic extract (pH 1.5) of opium or extracted alkaloid mixture, is obtained in the usual manner. This acidic fraction is extracted with chloroform, which removes a number of alkaloids including papaverine, narcotine, thebaine and laudanosine, where present. The acidic aqueous phase is then treated with dichloroacetic acid and fiuther extracted with chloroform. Morphine and codeine, where present, remain in the aqueous phase but a number of alkaloids, including (S)-reticuline, partition into the organic phase. The organic phase is subsequently evaporated to dryness and the residue dissolved in 0.1 M NaOH. Laudanine and laudanidine partition into the chloroform layer. The aqueous layer is treated with sodium bicarbonate and the resultant aqueous layer extracted with ether. The ether layer is found to contain (S)-reticuline.

### Example 5

### Analysis

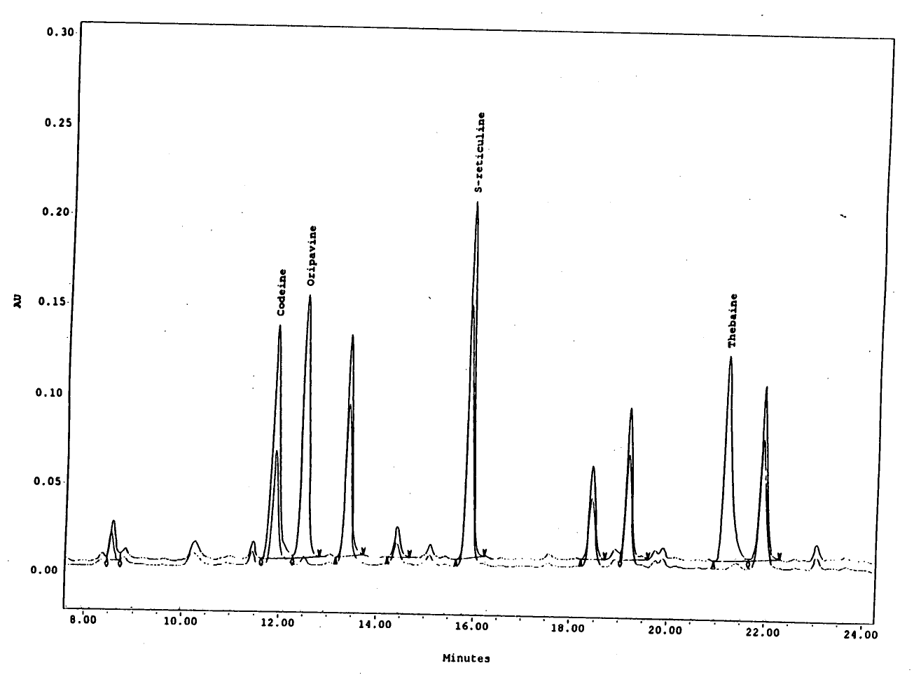

A HPLC trace of an E40R/41 R extract is shown in FIG. 1. The extract alone is the bottom trace, while the top trace is an solution containing extract and standards. (S)-reticuline is shown as having a retention time of about 16 minutes.

### Example 6

### Calculation

Phenanthrene alkaloids are those incorporating the phenanthrene ring system into their structure. Morphine is an example of such a phenanthrene type alkaloid. Reticuline however does not include this in its structure but has the “benzyl-isoquinoline” structure as its major structural element.

In the threshed straw of commercial poppies grown in Australia, (S)-reticuline constitutes no more than 0.04%, and the sum of all the phenanthrene alkaloids (morphine, codeine, thebaine and oripavine) is of the order of 1.2-2.7%, depending on the variety grown and factors such as crop nutrition and rainfall received.

Thus, 0.04/2.0×100=2%

In the reticuline poppies, the concentration of (S)-reticuline in the threshed poppy straw is about 2.5%, whereas the concentration of the sum of the phenanthrene alkaloids is at best 0.1%.

Thus, the percentage ratio is 2.5/0.1×100=2500%

### Example 7

### Improved Procedure for Extraction of Reticuline

An improved process for the isolation of crude reticuline was developed to generate an aqueous concentrate from poppy straw. The process was then optimised to obtain a product of improved purity.

The process flowchart with mass balances is represented in Scheme 4 below.

SCHEME 4

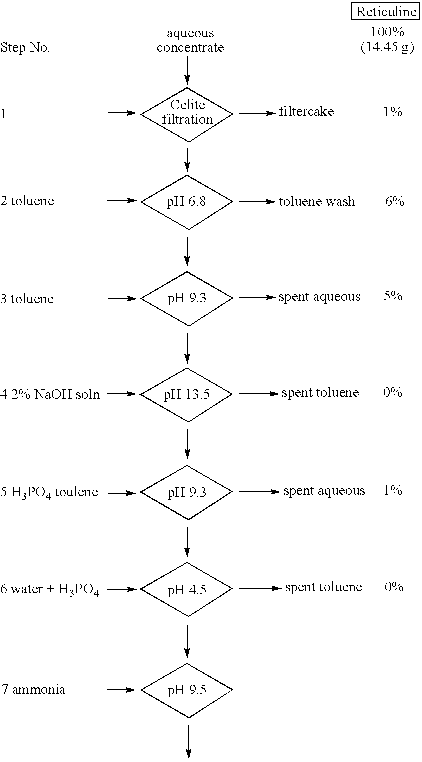

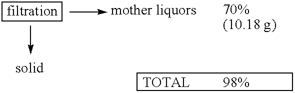

1. Concentrate Preparation

The dried ground straw was extracted with 80% ethanol at pH 4.5 (with acetic acid), and the resultant rich miscella was concentrated 8 fold under vacuum at 50° C. This miscella was produced batchwise by extracting straw in 100 gram lots with 1.0 litre of solvent and 50 mLs acid for 30 minutes at 40° C. Extraction efficiency was improved by using two countercurrent extractions. The miscella was adjusted to pH 6.0 with ammonia (˜30% w/w) prior to concentration by Buchi Rotavap, and the aqueous concentrate was filtered through a Celite bed.

2. Caustic Extraction of Toluene Solution

A toluene wash at pH 6.8, to remove levels of impurities, was applied to the concentrate prior to toluene extraction at pH 9.2. The toluene solution at pH 9.2 contained nearly all the available (S)-reticuline, rendering the aqueous solution spent.

Oripavine can be separated and isolated from a toluene solution containing both thebaine and oripavine by caustic extractions. This procedure was applied to the reticuline process, since reticuline has phenolic properties similar to oripavine. The resultant caustic extract was rich in reticuline and coloured black, but contained significantly reduced levels of impurities.

3. Removal of Coloured Impurities.

Attempts to precipitate a solid directly from the caustic extract by adjusting to pH 9.2 with phosphoric acid did not produce a crystalline solid. The resultant precipitate was a very sticky gum which did not disperse into a slurry. The caustic solution was therefore extracted with toluene at pH 9.2. The caustic solution (now spent of alkaloid) remained black, while the toluene solution of reticuline was almost colourless. This procedure affords an excellent means for the removal of a substantial amount of colour. An acid extraction of this toluene solution gave a relatively clean aqueous concentrate from which reticuline base can be precipitated.

4. Isolation of Extracted Alkaloid mixture

Dilute ammonia (˜80% w/w) was slowly added to the acidic reticuline solution to adjust the pH to 9.2 while maintaining the ambient temperature at 40° C. The slurry was aged for a few hours at ambient, and isolated by filtration. The cake was washed with two displacement volumes of water, and dried in vacuo at 50° C.

5. Assay Methodology

The HPLC method for analyses of these experiments is shown in Table I below. This isocratic method gives good separation between the main reticuline peak and the three major unknown components.

TABLE 1
HPLC assay method

| | |
|---|---|
| Mobile phase | 27% v/v methanol, in 0.8% triethylamine, |
| | to pH with H₃P0₄ |
| Flow rate | 1.0 mL/min |
| Wavelength | 284 nm |
| Column | Alltech Altima C18 |
| Retention times | reticuline: 10.1 minutes |

Scheme 5 below details the steps of a typical process.

### SCHEME 5

Part A: Straw Extraction.

1. Take reticuline straw which is dry, free of seed and ground to a fine powder.
2. Prepare a mixture consisting of 100 grams of ground straw, 1.0 litre of solvent (80% v/v ethanol) and 50 mLs acetic acid. Ensure the pH is in the range 4.3-4.8. Agitate at 40° C. for 30 minutes.
3. Filter, and put the filtrate (rich miscella) aside.
4. Take the filtered straw and extract with 1.0 litre fresh solvent and 50 mLs acetic acid (pH 4.3-4.8) at 40° C. for 30 minutes.
5. Filter, and discard the spent straw.
6. Extract a fresh lot of straw (100 g) with the filtrate from step 5, at 40° C. for 30 minutes.
7. Filter, and put the filtrate (rich miscella) aside. Extract the filtered straw with 1.0 litre fresh solvent and 50 mLs acetic acid at 40° C. for 30 minutes (as in step 40).
8. Repeat steps 5, 6 and 7 to process all the available straw.
9. Combine all the rich miscella and adjust the pH to 6.0-6.2 with ammonia (28% v/v).
   Part B: Concentration and Purification
1. Concentrate the rich miscelia under vacuum 8 to 10 fold. Do not exceed 60° C.
2. Filter the resulting concentrate through a bed of Celite, and wash the bed with a cake volume of warm water.
   Perform steps 3 to 17 at 40° C.
3. Add 0.3 volumes toluene, and adjust the pH to 6.8 using 40% w/v KOH or NaOH solution. Mix for 10 minutes, settle for 15 minutes. Separate the phases.
4. Perform a second toluene wash on the aqueous phase at pH 6.8, as in step 3. 5. Combine the toluene washes, and treat as spent.
6. Add 0.3 volumes of toluene to the aqueous phase, and adjust the pH to 9.3 using 40% w/v KOH solution. Mix for 10 minutes, settle for 15 minutes. Separate the phases.
7. Perform a second toluene extraction on the aqueous phase at pH 9.3, as in step 6.
8. Combine the toluene extracts, treat the aqueous phase as spent.
9. Add 0.3 volumes of a 2% w/v KOH solution to the toluene extracts. Mix for 10 minutes, settle for 15 minutes. Separate the phases.
10. Perform a second caustic extraction as in step 9, and combine the caustic extracts. Treat the toluene stream as spent solvent.
   (To minimise the time that reticuline is kept in highly alkaline conditions, this caustic solution should not be stored for a long period, ie not more than 8 hours).
11. Add 0.5 volumes toluene to the caustic extracts, and adjust the pH to 9.3 using concentrated H₃PO₄. Mix for 10 minutes, settle for 15 minutes. Separate the phases.
12. Perform a second toluene extraction at pH 9.3, as in step 11. 13. Combine the toluene extracts, treat the aqueous phase as spent.
14. Add 0.3 volumes water to the toluene extracts, and adjust the pH to 4.5 using concentrated H₃PO₄. Mix for 10 minutes, settle for 15 minutes. Separate the phases.
15. Perform a second extraction at pH 4.5, as in step 14. 16. Combine the acid extracts, treat the toluene phase as spent.
17. Slowly add 8% v/v ammonia to the acid extract to adjust the pH to 9.3. Ensure that agitation is sufficient to dissolve any localised precipitation, and adjust ammonia addition accordingly.
18. Age for 4-8 hours at ambient, filter, wash cake with two cake volumes of water, and dry the solid.
19. Extract the mother liquors with toluene (2×0.2 volumes) at pH 4.5. This toluene extract should be recycled to a later batch, or extracted into an aqueous acid solution for precipitation at pH 9.2.

The results of the process are summarised in Table 2 below.

TABLE 2
HPLC assay results

| | | | | | Reticuline (3) | | |
|---|---|---|---|---|---|---|---|
| | | | Colour of | Volume | | | % age |
| Step | pH | Sample | solution | (ml or g) | g/L | grams | yield |
| 1 | 5.5 | filtered concentrate | dark | 1200 | 12.04 (1) | 14.45 | 100% |
| | | filtercake | green | 100 | 0.76 | 0.08 | 1% |
| 2 | 6.8 | toluene wash | green | 550 | 1.61 | 0.89 | 6% |
| | | conc after wash | dark | 1500 | 9.04 | 13.56 | 94% |
| 3 | 9.3 | spent aqueous | dark | 1550 | 0.45 | 0.70 | 5% |
| | | toluene extract | yellow | 500 | 25.00 | 12.50 | 87% |
| 4 | 13.5 | spent toluene | colourless | 500 | 0.01 | 0.01 | 0% |
| | | caustic extract | black | 450 | 27.54 | 12.39 | 86% |
| 5 | 9.3 | spent aqueous | black | 430 | 0.25 | 0.11 | 1% |
| | | toluene extract | colourless | 400 | NOT | ASSA | YED |
| 6 | 4.5 | spent toluene | colourless | 400 | 0.00 | 0.00 | 0% |
| | | acid extract | light yellow | 400 | NOT | ASSA | YED |
| 7 | 9.3 | dried solid | creamy yellow | 11.8 | 86.3% | 10.18 | 70% |
| | | mother liquors | yellow | 500 | 4.25 | 2.13 | 15% |
| | | dried 2nd crop | yellow | 2.2 | 91.0% | 2.13 | 0.15 |
| | | solid (2) | | | | | |

Note:
(1) reticuline result for filtered concentrate based on combined results for step 2.
(2) 2nd crop was isolated after extraction with toluene at pH9.2, and evaporation of the extract to dryness.
(3) Concentrations of reticuline were calculated using a laudanine standard.

Accurate quantitation of reticuline was not possible due to the lack of a reticuline standard. The results in Table 2 are relative to a laudanine standard purified locally.

Precipitation of the crystalline crude reticuline base at pH 9.2 was very difficult due to gum formation. It was necessary to add the ammonia very slowly to allow localised precipitation to dissolve, and gum formation was minimised by adding dilute ammonia (3 fold dilution with water to 8-10% w/w) 25

It was observed that the relatively pure aqueous solutions of reticuline were dark yellow at pH>8, but light yellow in acidic conditions. A light-coloured acid solution of reticuline, therefore, gave rise to yellow coloured reticuline base solid.

The total quantity of crude reticuline (average assay 80%) obtained from all of 5 the available straw was 21.5 grams as dry weight.

The process summarised in Scheme 4 (described in detail in Scheme 5) represents a good method for the isolation of (S)-reticuline rich extracted alkaloid mixture from poppy straw. Implementation of this process on a large scale may require some minor alterations, such as the use of lime to treat the straw instead of acetic acid to reduce metal corrosion. This process could be scaled up to a factory with no specialised apparatus being necessary for the large scale extraction of reticuline.

This process is sufficient to produce (S)-reticuline product of at least 80% purity.

Further purification may be accomplished by use a co-solvent during precipitation or isolating a salt of reticuline, such as the bitartrate or the oxalate. The procedure described in Scheme 5 does not represent any major hazards other than those that currently exist in the morphine extraction process. No excessive temperatures or unusual solvents or reagents are required.

Although the invention has been described with reference to specific embodiments, modifications that are within the knowledge of those skilled in the art are also contemplated as being within the scope of the present invention.

1. A stably reproducing Papaver somniferum having an (s)-reticuline content higher than that of a native Papaver somniferum.

2. A stably reproducing Papaver somniferum, which upon the harvesting of the poppy capsules will yield a poppy straw having an (S)-reticuline content higher than the poppy straw obtained from a native Papaver somniferum.

3. A stably reproducing Papaver somniferum, which upon the collection and drying of the latex from the immature poppy capsules will yield an opium having an (S)-reticuline content higher than the latex obtained from a native Papaver somniferum.

4. A stably reproducing Papaver somniferum according to claim 1 in which the production or activity of (S)-reticuline oxidase is inhibited.

5. A stably reproducing Papaver somniferum according to claim 1 in which the production or activity of dehydroreticuline reductase is inhibited.

6. A stably reproducing Papaver somniferum according to claim 1 in which the production or activity of berberine bridge enzyme (BBE) is inhibited.

7. A stably reproducing Papaver somniferum according to claim 1 in which the production or activity of two or more enzymes selected from the group comprising: (S)-reticuline oxidase, dehydroreticuline reductase and berberine bridge enzyme (BBE) are inhibited.

8. A stably reproducing Papaver somniferum according to claim 2, which yields a poppy straw having an (S)-reticuline content greater than 1.0%.

9. A stably reproducing Papaver somniferum according to claim 3, which yields an opium having an (S)-reticuline content greater than 10%.

10. A stably reproducing Papaver somniferum according to claim 1 which upon the harvesting of the poppy capsules will yield a poppy straw, an opium or an extracted alkaloid mixture having an (S)-reticuline to phenanthrene alkaloid ratio of about 100% or greater.

11. A stably reproducing Papaver somniferum according to claim 1 which upon the harvesting of the poppy capsules will yield a poppy straw, an opium or an extracted alkaloid mixture having an (S)-reticuline to phenanthrene alkaloid ratio of about 200% or greater.

12. A stably reproducing Papaver somniferum according to claim 1 which upon the harvesting of the poppy capsules will yield a poppy straw, an opium or an extracted alkaloid mixture having an (S)-reticuline to phenanthrene alkaloid ratio of about 1250% or greater.

13. A stably reproducing Papaver somniferum according to claim 1 which upon the harvesting of the poppy capsules will yield a poppy straw, an opium or an extracted alkaloid mixture having an (S)-reticuline to phenanthrene alkaloid ratio of about 2500%.

14. A stably reproducing Papaver somniferum according to claim 1 which upon the harvesting of the poppy capsules will yield a poppy straw, an opium or an extracted alkaloid mixture having substantially no phenanthrene alkaloid content.

15. A seed yielding a stably reproducing Papaver somniferum according to claim 1.

16. Poppy straw of a stably reproducing Papaver somniferum, the threshed straw having an (S)-reticuline content higher than that of the poppy straw of a native Papaver somniferum.

17. Poppy straw according to claim 16, wherein the (S)-reticuline to phenanthrene alkaloid ratio is 100% or greater by weight.

18. Poppy straw according to claim 16, where in the (S)-reticuline to phenanthrene alkaloid ratio is 200% or greater by weight.

19. Poppy straw according to claim 16, wherein the (S)-reticuline to phenanthrene alkaloid ratio is 1250% or greater by weight.

20. Poppy straw according to claim 16, wherein the (S)-reticuline to phenanthrene alkaloid ratio is about 2500% by weight.

21. Poppy straw according to claim 16, having substantially no phenanthrene alkaloid content.

22. Poppy straw according to claim 16, having an (S)-reticuline content greater than 1.0%, and more preferabley greater than 2.0%.

23. Poppy straw according to claim 22, having an (S)-reticuline content of about 3-4%.

24. Opium of a stably reproducing Papaver somniferum, the opium having an (S)-reticuline content higher than that of the opium of a native Papaver somniferum.

25. Opium according to claim 24, wherein the (S)-reticuline to phenanthrene alkaloid ratio is 100% or greater by weight.

26. Opium according to claim 24, wherein the (S)-reticuline to phenanthrene alkaloid ratio is 200% or greater by weight.

27. Opium according to claim 24, wherein the (S)-reticuline to phenanthrene alkaloid ratio is 1250% or greater by weight.

28. Opium according to claim 24, wherein the (S)-reticuline to phenanthrene alkaloid ratio is about 2500% by weight.

29. Opium according to claim 24, having substantially no phenanthrene alkaloid content.

30. Opium according to claim 24, having an (S)-reticuline content greater than 10%.

31. Extracted alkaloid mixture of a stably reproducing Papaver somniferum, the extracted alkaloid mixture having an (S)-reticuline content higher than that of the extracted alkaloid mixture of a native Papaver somniferum.

32. Extracted alkaloid mixture according to claim 31, wherein the (S)-reticuline to phenanthrene alkaloid ratio is 100% or greater by weight.

33. Extracted alkaloid mixture according to claim 31, wherein the (S)-reticuline to phenanthrene alkaloid ratio is 200% or greater by weight.

34. Extracted alkaloid mixture according to claim 31, wherein the (S)-reticuline to phenanthrene alkaloid ratio is 1250% or greater by weight.

35. Extracted alkaloid mixture according to claim 31, wherein the. (S)-reticuline to phenanthrene alkaloid ratio is 2500% or greater by weight.

36. Extracted alkaloid mixture according to claim 31, having substantially no phenanthrene alkaloid content.

37. Extracted alkaloid mixture according to any one of claims claim 31 to 36 claim 31, having an (S)-reticuline content greater than 30%, and more preferably greater than 60%.

38. A stand of a stably reproducing Papaver somniferum according to any one of claims 1 to 14 claim 1.

39. (S)-reticuline when obtained from a stably reproducing Papaver somniferum according to claim 1.

40. A method for the production of (S)-reticuline which comprises the steps of:
a) harvesting poppy capsules of a stably reproducing Papaver somniferum to produce a straw, wherein the straw has a higher (S)-reticuline content than the straw of a native Papaver somniferum, and
b) chemically extracting the (S)-reticuline from the straw.

41. A method for the production of (S)-reticuline which comprises the steps of:
a) collecting and drying the latex of the immature poppy capsules of a stably reproducing Papaver somniferum to produce opium wherein the opium has a (S)-reticuline content higher than that of the opium of a native Papaver somniferum and
b) chemically extracting the (S)-reticuline from the opium.

42. A method according to claim 40 or claim 41, wherein the stably reproducing Papaver somniferum yields a poppy straw having an (S)-reticuline content greater than 1.0% and more preferably greater than 2.0%.

43. A method according to claim 40 or claim 41, wherein the stably reproducing Papaver somniferum yields an opium having an (S)-reticuline content greater than 10%.

44. (S)-reticuline when obtained by a method according to claim 40.

45. A method to improve the (S)-reticuline yield of a stably reproducing Papaver somniferum, the method comprises the steps of:
a) exposing at least one poppy seed of Papaver somniferum to a mutagenising agent,
b) growing the at least one poppy seed to produce a plant bearing a leaf or an immature poppy capsule, optionally through multiple self fertilized generations,
c) sampling the leaf or poppy capsule for the presence of (S)-reticuline, morphine and codeine, and
d) repeating steps a) to c) until a poppy plant of Papaver somniferum is obtained having a (S)-reticuline content higher than that of a native Papaver somniferum.

46. A method according to claim 45, wherein steps a) to c) are repeated until the (S)-reticuline content shows no further increase on mutagenesis.

47. (canceled)

48. (canceled)

49. (canceled)

50. Method for purifying reticuline from an aqueous extract of poppy straw comprising the following steps:
(i) mix said extract with toluene at near neutral pH and separate the aqueous and the non-aqueous phases,
(ii) mix aqueous phase from step (ii) with toluene at pH of about 9.0 to about 9.4 and separate the aqueous and the non-aqueous phases,
(iii) extract reticuline from the non-aqueous phase by caustic extraction.

51. A method according to claim 50, wherein step (i) is repeated before proceeding to step (ii).

52. A method according to claim 50 or claim 51, wherein step (ii) is repeated before proceeding to step (iii).

53. A method according to claim 50, wherein pH in step (i) is about 6.8.

54. A method according to claim 50, wherein pH in step (ii) is about 9.3.

55. A method according to claim 50, further comprising
(iv) mixing caustic extract of step (iii) with toluene at pH of about 8.5 to about 9.5 and separating the aqueous and the non-aqueous phases,
(v) mixing the non-aqueous phase from step (iv) with water at acidic pH, and separating the aqueous and the non-aqueous phases,
(vi) adding alkali to aqueous phase at ambient temperature, ageing for a time sufficient to induce formation of a precipitate and collecting precipitate containing reticuline.

56. A method according to claim 55, wherein steps (iv) and/or (v) are repeated.

57. A method according to claim 55, wherein pH in step (iv) is about 9.3.

58. A method according to claim 56, wherein pH in step (v) is about 4.5.

59. A method according to claim 56, wherein alkali is added to achieve a pH of about 9.3.

60. (S)-reticuline obtained by a method according to claim 51.
